(19) United States
(12) Patent Application Publication
(10) Pub. No.: US 2025/0226089 A1
(43) Pub. Date: Jul. 10, 2025

(54) APPARATUS AND METHOD FOR GENERATING AN ELECTROCARDIOGRAM VERIFICATION SET

(71) Applicant: anumana, Inc., Cambridge, MA (US)

(72) Inventor: Rakesh Barve, Bengaluru (IN)

(73) Assignee: anumana, Inc., Cambridge, MA (US)

(21) Appl. No.: 18/403,840

(22) Filed: Jan. 4, 2024

(51) Int. Cl. G16H 50/20

(52) U.S. Cl. CPC G16H 50/20

(57) ABSTRACT

An apparatus for generating an electrocardiogram verification set is disclosed. The apparatus includes processor and a memory communicatively connected to the processor. The memory instructs the processor to receive digital ECG data. The memory instructs the processor to convert the digital ECG data into analog ECG data. The memory instructs the processor to generate an ECG validation set as a function of the analog ECG data. The memory instructs the processor to validate a diagnostic machine learning model as a function of the ECG validation set. Validating the diagnostic machine learning model includes iteratively training the diagnostic machine learning model using diagnostic training data. Validating the diagnostic machine learning model includes generating performance data based on the ECG validation set. Validating the diagnostic machine learning model includes accepting the diagnostic machine learning model as a function of a comparison between the performance data and a validation threshold.

### FIELD OF THE INVENTION

The present invention generally relates to the field of artificial intelligence. In particular, the present invention is directed to an apparatus and a method for generating an electrocardiogram verification set.

### BACKGROUND

Validating machine learning models with using data generated from patients has proven to be a laborious and time-consuming task. This is partially attributed to issues associated with the consistency and format of the electrocardiogram signals. This is particularly an issue when the electrocardiogram signals are generated from various origins.

### SUMMARY OF THE DISCLOSURE

In an aspect, an apparatus for generating an electrocardiogram verification set is disclosed. The apparatus includes a processor and a memory communicatively connected to the processor. The memory instructs the processor to receive digital ECG data. The memory instructs the processor to convert the digital ECG data into analog ECG data. The memory instructs the processor to generate an ECG validation set as a function of the analog ECG data. The memory instructs the processor to validate a diagnostic machine learning model as a function of the ECG validation set. Validating the diagnostic machine learning model includes iteratively training the diagnostic machine learning model using diagnostic training data. Validating the diagnostic machine learning model includes generating performance data based on the ECG validation set. Validating the diagnostic machine learning model includes accepting the diagnostic machine learning model as a function of a comparison between the performance data and a validation threshold.

In another aspect, a method for the generating an electrocardiogram verification set is disclosed. The method includes receiving, using at least a processor, digital ECG data. The method includes converting, using the at least a processor, the digital ECG data into analog ECG data. The method includes generating, using the at least a processor, an ECG validation set as a function of the analog ECG data. The method includes validating, using the at least a processor, a diagnostic machine learning model as a function of the ECG validation set. Validating the diagnostic machine learning model includes iteratively training the diagnostic machine learning model using diagnostic training data. Validating the diagnostic machine learning model includes generating performance data based on the ECG validation set. Validating the diagnostic machine learning model includes accepting the diagnostic machine learning model as a function of a comparison between the performance data and a validation threshold.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for generating an electrocardiogram verification set;

FIG. 2 is a block diagram of an exemplary machine-learning process;

FIG. 3 is a block diagram of an exemplary embodiment of a validation database;

FIG. 4 is a diagram of an exemplary embodiment of a neural network;

FIG. 5 is a diagram of an exemplary embodiment of a node of a neural network;

FIG. 6 is an illustration of an exemplary embodiment of fuzzy set comparison;

FIG. 7 is a flow diagram of an exemplary method for generating an electrocardiogram verification set; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and a method for generating an electrocardiogram verification set is disclosed. The apparatus includes processor and a memory communicatively connected to the processor. The memory instructs the processor to receive digital ECG data. The memory instructs the processor to convert the digital ECG data into analog ECG data. The memory instructs the processor to generate an ECG validation set as a function of the analog ECG data. The memory instructs the processor to validate a diagnostic machine learning model as a function of the ECG validation set. Validating the diagnostic machine learning model includes iteratively training the diagnostic machine learning model using diagnostic training data. Validating the diagnostic machine learning model includes generating performance data based on the ECG validation set. Validating the diagnostic machine learning model includes accepting the diagnostic machine learning model as a function of a comparison between the performance data and a validation threshold. Exemplary embodiments illustrating aspects of the present disclosure are described below in the context of several specific examples.

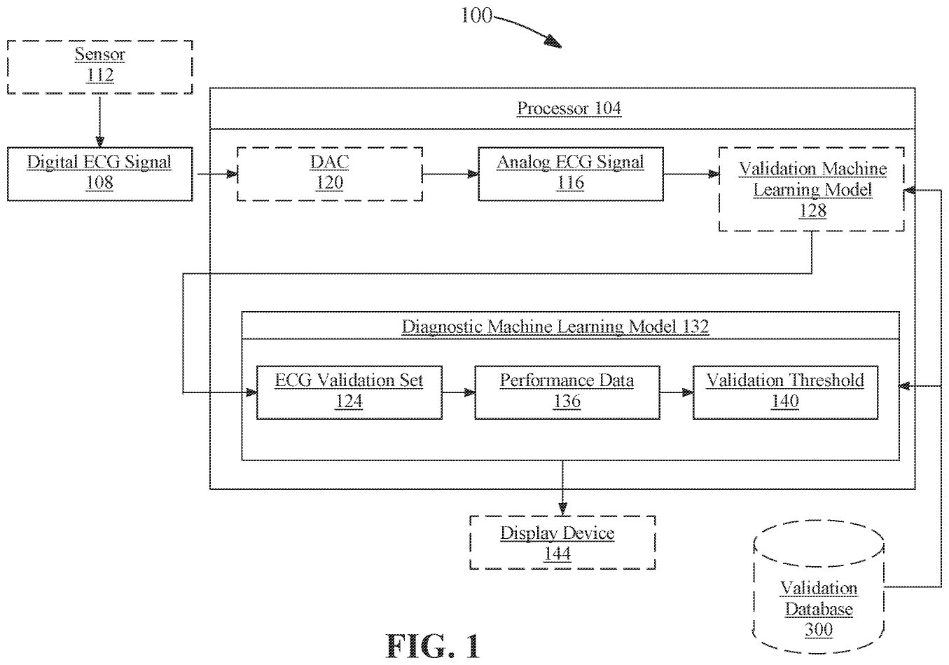

Referring now to FIG. 1, an exemplary embodiment of an apparatus 100 for generating an electrocardiogram verification set is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 includes a memory. Memory is communicatively connected to processor 104. Memory may contain instructions configuring processor 104 to perform tasks disclosed in this disclosure. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, apparatus, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example, and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, without limitation, via a bus or other facility for intercommunication between elements of a computing device. Communicative connecting may also include indirect connections via, for example, and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, processor 104 is configured to receive digital ECG data 108. As used in the current disclosure, “digital ECG data” refers to the digital representation of the electrical activity of the heart recorded over time. Digital ECG data 108 may include a plurality of ECG signals represented in a digital format. As used in the current disclosure, a “electrocardiogram signal” is a signal representative of electrical activity of heart. The ECG signal may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves provide information about the duration and regularity of various phases of the cardiac cycle. The ECG signal can help diagnose various heart conditions, such as arrhythmias, myocardial infarction (heart attack), conduction abnormalities, and electrolyte imbalances.

With continued reference to FIG. 1, digital ECG data 108 is often stored in various file formats. This may include formats such as DICOM, HL7, or simple text-based formats. These files contain the time and voltage measurements, patient information, and metadata. In an embodiment, digital ECG data 108 may be received from a database, application program interface (API), electronic health records, and the like. As used in the current disclosure, “electronic health records” are digital records containing a patient's medical history, diagnoses, medications, treatment plans, and other relevant information. EHRs are used by healthcare providers to track and manage patient care. In an embodiment, digital ECG data 108 may include a plurality of metadata. As used in the current disclosure, “metadata” refers to descriptive or informational data that provides details about the digital ECG data 108. Metadata may include descriptive metadata, wherein descriptive metadata is configured to describe the content, context, and structure of the data. In an embodiment, metadata may include data regarding the lead system the digital ECG data 108 was recording. ECGs are typically recorded using multiple leads, each of which provides a different view of the heart's electrical activity. Common lead systems include the 12-lead, 6-lead, 3-lead, and single-lead ECGs. The specific lead system used to generate the digital ECG data 108 and their configurations may be documented in the metadata. In some embodiments, metadata associated with the digital ECG data 108 may include information such as time, geographic location, medical facility names, medical professional logs, patient names, patient IDs, patient data, along with any other patient specific data. Metadata may be used to describe records of how the data has been accessed, utilized, or modified over time, aiding in understanding data usage patterns, and optimizing access.

With continued reference to FIG. 1, processor 104 may be configured to receive digital ECG data 108 using an application programming interface (API). As used herein, an “application programming interface” is a set of functions that allow applications to access data and interact with external software components, operating systems, or microdevices, such as another web application or computing device. An API may define the methods and data formats that applications can use to request and exchange information. APIs enable seamless integration and functionality between different systems, applications, or platforms. An API may deliver digital ECG data 108 to apparatus 100 from a system/application that is associated with a user, medical provider, or other third party custodian of user information. An API may be configured to query for web applications or other websites to retrieve digital ECG data 108. An API may be further configured to filter through web applications according to a filter criterion. In this disclosure, “filter criteria” are conditions the web applications must fulfill in order to qualify for API. Web applications may be filtered based off these filter criteria. Filter criterion may include, without limitation, web application dates, web application traffic, web application types, web applications addresses, and the like. Once an API filters through web applications according to a filter criterion, it may select a web application. Processor 104 may transmit, through the API, digital ECG data 108 to apparatus 100. API may further automatically fill out user entry fields of the web application with the user credentials in order to gain access to the digital ECG data 108. Web applications may include, without limitation, a medical database, hospital website, file scanning, email programs, third party websites, governmental websites, or the like.

With continued reference to FIG. 1, the plurality of electrocardiogram signals may capture a temporal view of cardiac electrical activities. A “temporal view,” as used in the current disclosure, refers to the analysis and visualization of heart-related events and phenomena over time. A temporal view may include patterns, changes, and dynamics of cardiac activity over time. A temporal view may include information surrounding the rhythm of the heart, including the regularity or irregularity of heartbeats. It allows for the identification of various rhythm abnormalities such as tachycardia (fast heart rate), bradycardia (slow heart rate), or arrhythmias (irregular heart rhythms). A temporal view of cardiac activities in three dimensions may refer to a visualization that represents the temporal evolution of cardiac events or phenomena in a three-dimensional space. It provides a comprehensive understanding of how various cardiac activities change over time. The ECG signal 108 may move through the 3D space of the heart over time. The signal does not just move forward in time, it also moves through the physical space of the heart, from SA node through atria, to AV node, and then through the ventricles. Such movement of the electrical signal through the heart's physical space over time can be referred to as “spatiotemporal excitation and propagation” which could be captured by plurality of ECG signals. It is a way of observing and analyzing the timing and sequence of the heart's electrical activity as it moves through the physical structure of the heart. In the current case the dimensions may include axes representing time, spatial dimensions, and cardiac activity. By combining the temporal, spatial, and cardiac activity dimensions, the temporal view of cardiac activities in three dimensions allows for a comprehensive visualization and analysis of dynamic changes occurring within the heart. It can be used to study phenomena like electrical conduction, ventricular wall motion, valve function, blood flow dynamics, or the interaction between different regions of the heart. This visualization approach provides valuable insights into the complex temporal dynamics of cardiac activities and aids in understanding cardiac function, pathology, and treatment evaluation.

With continued reference to FIG. 1, processor 104 may be configured to anonymize the digital ECG data 108 and its accompanying metadata using an anonymization process. As used in the current disclosure, an “anonymization process” is the process of anonymizing personally identifiable information within the data. Digital ECG data 108 may be a crucial step in preserving privacy and ensuring compliance with data protection regulations such as GDPR or HIPAA when developing machine learning models. Anonymization involves removing or obfuscating personally identifiable information (PII) and sensitive data while retaining the utility and quality of the data for model training. As used in the current disclosure, “personally identifiable information” refers to information used to identify and distinguish individual patients in healthcare records and systems. PII may include any identifiers described in the Health Insurance Portability and Accountability Act (HIPAA). Examples of PII include the name, address, phone number, email address, phone number, email address, social security number (SSN), national identification number, medical record number, health insurance information, beneficiary information, account numbers, and the like. Processor 104 may be configured to anonymize each patient identifier within digital ECG data 108 to ensure that no patient can be identified based on this data. In an embodiment, an anonymization processes may include redacting the patient identifiers within the digital ECG data 108. Redacting may be done using various methods like blacking out, using placeholders, or applying software tools to mask or replace the sensitive data. In an embodiment, this may involve removing or replacing patient identifiers with pseudonyms and/or generic terms. In another embodiment, the anonymization process may replace PII with pseudonyms or tokens. For example, processor 104 may replace names with unique identifiers, such as “User12345,” and email addresses with placeholders like user@email.com. In some cases, processor 104 may group data into broader categories to reduce the granularity of information. For instance, processor 104 can generalize ages into age groups (e.g., 20-30, 31-40) rather than using exact ages. In some cases, anonymization process may create synthetic data that mimics the statistical properties of the original data. This can help maintain data utility while preventing re-identification.

With continued reference to FIG. 1, metadata may be detached from the digital ECG data 108 using various methods and techniques. Detaching metadata from digital ECG data 108 is the process of separating identifying or contextual information from the core content or information provided by users. This practice may be useful for safeguarding individual privacy and data anonymity. By removing metadata, such as geolocation, timestamps, or other PII, from the associated data, it becomes significantly more challenging to trace the information back to specific individuals, thus reducing the risk of unauthorized surveillance or misuse of personal information. Detaching metadata may protect user privacy and maintaining data integrity in an increasingly interconnected digital world. In some cases, data profiling tools automatically analyze the dataset to detach metadata, including statistical summaries (e.g., min, max, mean, standard deviation), data distributions, unique values, and data quality metrics. Profiling tools can help understand the data's characteristics and identify anomalies. Additionally, software tools may be used to analyze the data to infer its underlying schema or structure. This process involves identifying data types, keys, relationships, and constraints based on patterns and regularities within the dataset. It is especially useful for unstructured or semi-structured data. In a non-limiting embodiment, natural language processing techniques can be used to extract metadata such as keywords, entities, topics, and sentiment analysis. NLP algorithms can automatically annotate and categorize text, providing valuable metadata about the content. Language processing techniques are discussed in greater detail hereinbelow. In an additional embodiment, machine learning algorithms can be trained to identify and extract specific metadata elements from the dataset. For example, a model could be trained to recognize dates, names, or numerical values within the dataset. For datasets sourced from the web, web scraping techniques, mentioned herein above, can be employed to extract metadata from web pages. This could include extracting information about the source, publication date, author, or any other relevant metadata present on the web. In a third embodiment, metadata may be extracted from the digital ECG data 108 utilizing APIs and data catalogs associated with specific datasets or data sources can provide standardized metadata. APIs may offer programmatic access to metadata and dataset information.

With continued reference to FIG. 1, processor 104 may be configured to identify personally identifiable information using a natural language processing model. As used in the current disclosure, a “natural language processing (NLP) model” is a computational model designed to process and understand human language. It leverages techniques from machine learning, linguistics, and computer science to enable computers to comprehend, interpret, and generate natural language text. The NLP model may preprocess the textual data, wherein the input text may include all text contained within the digital ECG data 108 its associated metadata, or any other data mentioned herein. Preprocessing the input text may involve tasks like tokenization (splitting text into individual words or sub-word units), normalizing the text (lowercasing, removing punctuation, etc.), and encoding the text into a numerical representation suitable for the model. The NLP model may include transformer architecture, wherein the transformers are deep learning models that employ attention mechanisms to capture the relationships between words or sub-word units in a text sequence. They consist of multiple layers of self-attention and feed-forward neural networks. The NLP model may weigh the importance of different words or sub-word units within a text sequence while considering the context. It enables the model to capture dependencies and relationships between words, considering both local and global contexts. This process may be used to identify a plurality of personally identifiable information. Language processing model may include a program automatically generated by processor 104 and/or named entity recognition system to produce associations between one or more significant terms extracted from the digital ECG data 108 its associated metadata and detect associations, including without limitation mathematical associations, between such significant terms. Associations between language elements, where language elements include for purposes herein extracted significant terms, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted significant term indicates a given category of semantic meaning. As a further example, statistical correlations and/or mathematical associations may include probabilistic formulas or relationships indicating a positive and/or negative association between at least an extracted significant term and/or a given semantic relationship; positive or negative indication may include an indication that a given document is or is not indicating a category semantic relationship. Whether a phrase, sentence, word, or other textual element in the digital ECG data 108 its associated metadata constitutes a positive or negative indicator may be determined, in an embodiment, by mathematical associations between detected significant terms, comparisons to phrases and/or words indicating positive and/or negative indicators that are stored in memory at processor 104, or the like.

With continued reference to FIG. 1, the plurality of electrocardiogram signals may be generated using at least a sensor 112. As used in this disclosure, a “sensor” is a device that is configured to detect an input and/or a phenomenon and transmit information related to the detection. Sensor 112 may detect a plurality of data. A plurality of data detected by sensor 11 may include, but is not limited to, electrocardiogram signals, heart rate, blood pressure, electrical signals related to the heart, and the like. In one or more embodiments, and without limitation, sensor 112 may include a plurality of sensors 112. In one or more embodiments, and without limitation, sensor 112 may include one or more electrodes, and the like. Electrodes used for an electrocardiogram (ECG) are small sensors or conductive patches that are placed on specific locations on the body to detect and record the electrical signals generated by the heart. Sensor 112 serves as the interface between the body and the ECG machine, allowing for the measurement and recording of the heart's electrical activity. A plurality of sensors 112 may include 10 electrodes used for a standard 12-lead ECG, placed in specific positions on the chest and limbs of the patient. These electrodes are typically made of a conductive material, such as metal or carbon, and are connected to lead wires that transmit the electrical signals to the ECG machine for recording. Proper electrode placement is crucial to ensure accurate signal detection and recording.

With continued reference to FIG. 1, the plurality of sensors 112 may be placed on each limb, wherein there may be at least one sensor 112 on each arm and leg. These sensors 112 may be labeled I, II, III, V1, V2, V3, V4, V5, V6, and the like. For example, Sensor I may be placed on the left arm, Sensor II may be placed on the right arm, and Sensor III may be placed on the left leg. Additionally, a plurality of sensors 112 may be placed on various portions of the patient's torso and chest. For example, a sensor V1 may be placed in the fourth intercostal space at both the right sternal borders and sensor V2 may be fourth intercostal space at both the left sternal borders. A sensor V3 may also be placed between sensors V2 and V4, halfway between their positions. Sensor V4 may be placed in the fifth intercostal space at the midclavicular line. Sensor V5 may be placed horizontally at the same level as sensor V4 but in the anterior axillary line. Sensor V6 may be placed horizontally at the same level as V4 and V5 but in the midaxillary line.

With continued reference to FIG. 1, the plurality of sensors 112 may include augmented unipolar sensors. These sensors 112 may be labeled as aVR, aVL, and aVF. These sensor may be derived from the limb sensors and provide additional information about the heart's electrical activity. These leads are calculated using specific combinations of the limb leads and help assess the electrical vectors in different orientations. For example, aVR may be derived from Sensor II and Sensor III. In another example, aVL may be derived from sensor I and Sensor III. Additionally, a VF may be derived from Lead I and Lead II. The combination of limb sensors, precordial sensors, and augmented unipolar sensors allows for a comprehensive assessment of the heart's electrical activity in three dimensions. These leads capture the electrical signals from different orientations, which are then transformed into transformed coordinates to generate vectorcardiogram (VCG) representing magnitude and direction of electrical vectors during cardiac depolarization and repolarization. Transformed coordinates may include one or more a Cartesian coordinate system (x, y, z), polar coordinate system (r, θ), cylindrical coordinate system (ρ, φ, z), or spherical coordinate system (r, θ, φ). In some cases, transformed coordinates may include an angle, such as with polar coordinates, cylindrical coordinates, and spherical coordinates. In some cases, VCG may be normalized thus permitting full representation with only angles, i.e., angle traversals. In some cases, angle traversals may be advantageously processed with one or more processes, such as those described below and/or spectral analysis.

With continued reference to FIG. 1, processor 104 is configured to convert the digital ECG data 108 into analog ECG data 116. As used in the current disclosure, “analog ECG data” refers to an analog representation of the electrical activity of the heart recorded over time. Analog ECG data 116 may include a plurality of ECG signals represented in an analog format. As used in the current disclosure, an “analog format” refers to a method of representing information or data using continuous and continuously variable physical quantities, such as electrical voltage. Analog ECG data 116 may refer to the traditional method of recording and storing ECG signals in an analog, non-digital format. Analog ECG data involves the use of paper or other analog recording mediums to graphically represent the electrical activity of the heart. In some embodiments, the analog electrical signals from the heart are recorded directly onto special ECG paper. This paper typically has a grid pattern with horizontal and vertical lines, which helps in measuring the time and voltage of the ECG waveforms. Similar to digital ECG data 108, analog ECG data 116 also may use electrodes placed on the patient's skin. The electrodes may be connected to leads that transmit the electrical signals to the recording device. Different lead configurations may be used, such as the 12-lead system, 6-lead system, 3-lead system, etc., each providing a different perspective of the heart's electrical activity. Analog ECG data 116 may reflect electrical activity of the heart is continuously recorded as a series of analog waveforms on the ECG paper. The most important components of these waveforms include the P-wave, QRS complex, and T-wave, which represent different phases of the cardiac cycle.

With continued reference to FIG. 1, analog ECG data 116 may be recorded and stored on various physical mediums. Analog ECG data 116 may be stored on ECG paper. ECG machines printed the electrical waveforms directly onto a roll or sheets of special ECG paper. The resulting ECG tracings were a graphical representation of the heart's electrical activity over time. These paper printouts were often physically archived in patient medical records. Analog ECG data 116 could also be recorded and stored on magnetic tape. Specialized magnetic tape recorders may be used to capture the analog signal, which could then be played back for analysis. This medium allowed for easier archiving and retrieval compared to paper. In some cases, analog ECG data 116 may be recorded onto analog audio cassette tapes. While primarily designed for audio, these tapes could be adapted for ECG signal storage, especially in ambulatory monitoring systems. The signal could be played back and analyzed using dedicated equipment. Analog ECG data 116 could be stored on photographic film, similar to how images are stored on film. These films used light exposure to capture the ECG waveforms and could be processed and printed for analysis or archiving. In a few cases, analog ECG data 116 may be stored on analog magnetic disk drives, similar to early computer storage systems. These systems were less common for ECG signal storage but were used in some research or specialized applications.

With continued reference to FIG. 1, processor 104 may be configured to generate normalized ECG data as a function of the conversion of digital ECG data 108 to analog ECG data 116. Normalization in as it relates to ECG data refers to the process of adjusting or standardizing the amplitude, lead configuration, and other aspects of ECG signals to ensure consistency and comparability across different recordings, devices, and healthcare settings. As used in the current disclosure, a “normalized ECG data” is ECG data that has adjusted into a standardized format. A standardized format may be standard as it relates to the amplitude, lead system, recoding device, and the like. Generating normalized ECG data may include selecting a standard lead system to which the ECG data will be normalized. This may include 12-lead ECG, 6-lead ECG, 3-lead ECG, 1-Lead ECG, bipolar limb leads, unipolar limb leads, precordial chest leads, Lewis lead system, Goldberger leads, Frank leads, Wilson central terminal, mason-likar lead configuration, extended limb leads, and the like. In an embodiment, processor 104 may select a 12-lead ECG because it is the most widely used standard, as it provides a comprehensive view of the heart's electrical activity from multiple angles. The ECG data recorded in the original lead system, as indicated by the meta data, may be mathematically transformed into the equivalent signals of the standard lead system. This transformation can involve the combination of signals from various leads to derive the standard 12-lead configuration. In order to normalize the ECG data processor 104 may resample the ECG Data at a standard rate to ensure that the timing and frequency characteristics of the signal are consistent with the selected lead system. Common sampling rates for ECG data are typically in the range of 250 to 1000 samples per second (Hz). In some embodiments, ECG signals may be normalized with respect to their voltage amplitudes. Amplitude calibration ensures that the voltages in the ECG signals align with a standard reference, typically in millivolts (mV). In other embodiments, a normalization step may include adjusting the baseline of the ECG signals to a standard reference point, such as the isoelectric line (baseline) for the P-wave. This helps in ensuring that the baseline is consistent across different recordings.

With continued reference to FIG. 1, processor 104 may convert the digital ECG data 108 into analog ECG data 116 using a digital-to-analog converter (DAC) 120. As used in the current disclosure, a “digital-to-analog converter” is a device or component that converts digital signals, which are represented as binary numbers, into analog signals. This conversion is essential for generating a normalized representation of the digital ECG data 108 in an analog format. The inputs into the DAC 120 may include digital ECG data 108, typically in the form of binary values, which represent discrete voltage levels or samples of the ECG signal. This data is obtained from digital ECG recording devices or systems. The resolution of the DAC 120 is an important parameter, determining the precision of the analog signal it can generate. It's specified in terms of the number of bits (e.g., 12-bit, 16-bit, etc.). A higher bit count provides greater precision and a finer representation of the digital ECG signal 108 in an analog format. The DAC 120 may have a suitable sampling rate, which determines how quickly it can output analog samples per second. The sampling rate may match the digital ECG data's 108 rate to accurately reconstruct the analog signal. The DAC 120 should be able to produce analog signals within the required voltage range for ECG (typically in millivolts or microvolts). This ensures that the analog signal is compatible with ECG monitoring equipment and displays. The DAC may filter the analog ECG signals 116 to remove high-frequency noise and artifacts. This may include low-pass filtering to achieve this. The DAC 120 may require a reference voltage to establish the full-scale output range. It may use a precision voltage reference source to ensure accurate and stable conversions. A reference voltage may be included within the metadata associated with the digital ECG data 108. The DAC 120 may take the digital values of the ECG samples and converts them into analog voltage levels or analog ECG data 116. This is usually done using one or more DAC architectures, such as binary-weighted resistor DAC, R-2R ladder DAC, or delta-sigma DAC. The output of the DAC 120 is an analog voltage signal or analog ECG data 116 that closely resembles the original ECG waveform. This signal can be used for display on analog ECG monitors, for further analog processing, or for recording on analog mediums like paper.

With continued reference to FIG. 1, processor 104 is configured to calculate a confidence interval associated with the conversion of the analog ECG data. As used in the current disclosure, a “confidence interval” is a statistical range that provides an estimate of the likely range of values within which the true accuracy of the conversion process lies. A confidence interval may quantify the uncertainty associated with the accuracy assessment and provides a level of confidence regarding the precision of the conversion. When converting digital ECG data 108 to analog ECG data 116, it is desirable to evaluate how accurately the analog signal represents the original digital signal. Accuracy here refers to the extent to which the analog output faithfully replicates the digital input. A confidence interval may be a statistical concept that provides a range of values within which a population parameter (in this case, the accuracy of the conversion) is likely to fall. It is expressed with a specified level of confidence, often denoted by a percentage (e.g., 95% confidence interval). To calculate a confidence interval for the accuracy of the conversion, processor 104 may perform accuracy testing, which may involve comparing the analog ECG data 116 to the original digital ECG data 108. The confidence interval may be calculated based on the results of this testing. The confidence interval is associated with a chosen level of confidence, which represents the probability that the true accuracy of the conversion falls within the interval. In a non-limiting example, levels of confidence may include 95% and 99%, indicating that processor 104 is 95% or 99% confident that the true accuracy falls within the calculated range. Confidence intervals can also be used to estimate the measurement error introduced during the conversion process. This helps assess the precision of the conversion. In some embodiments, confidence intervals can also be used to estimate the measurement error introduced during the conversion process. This helps assess the precision of the conversion. In an embodiment, the confidence interval can be visually represented as an error bar on a graph or plot of the converted analog ECG data 116. The error bar extends above and below the converted signal, indicating the range of values within which the true analog signal is likely to lie with a specified level of confidence. The length of the error bar or the width of the confidence interval provides information about the uncertainty associated with the conversion. A wider error bar implies greater uncertainty, while a narrower error bar indicates higher confidence in the accuracy of the conversion. The interpretation of the error bar's width may be essential for understanding the clinical significance of the converted analog ECG data 116. A narrow error bar suggests that the conversion process is precise and reliable, while a wide error bar indicates the need for caution in interpreting the analog signal.

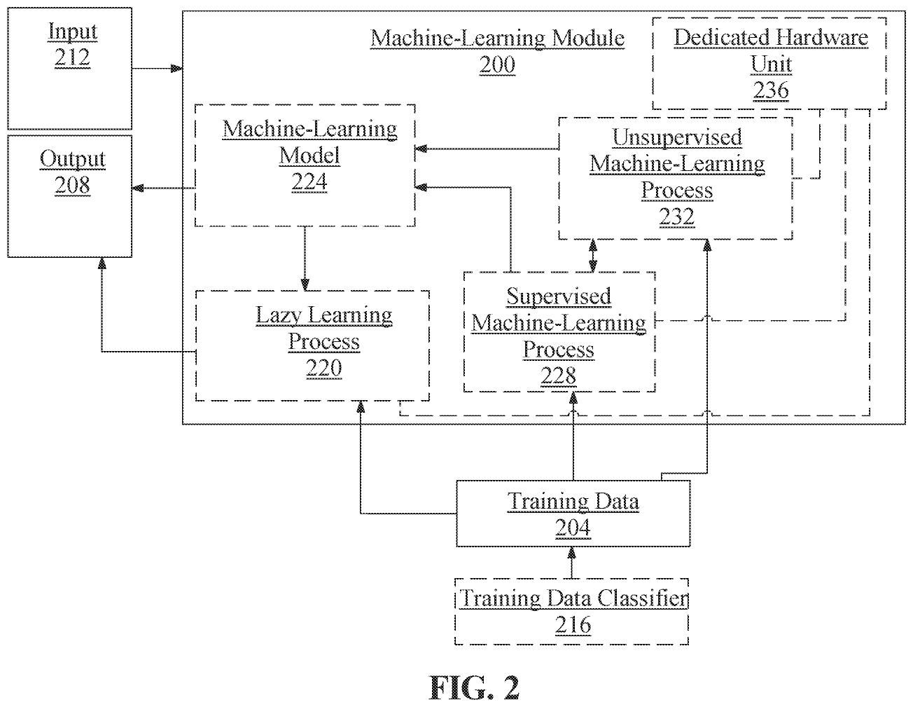

With continued reference to FIG. 1, processor 104 may generate a confidence interval associated with the conversion using a confidence machine-learning model. As used in the current disclosure, a “confidence machine-learning model” is a machine-learning model that is configured to generate a confidence interval associated with the conversion of digital ECG data 108 to analog ECG data 116. A confidence machine-learning model may be consistent with the machine-learning model described below in FIG. 2. Inputs to the confidence machine-learning model may include digital ECG data 108, analog ECG data 116, normalized ECG data, examples of confidence intervals, and the like. Outputs to the confidence machine-learning model may include confidence intervals tailored to the conversion of digital ECG data 108 to analog ECG data 116. Confidence training data may include a plurality of data entries containing a plurality of inputs that are correlated to a plurality of outputs for training a processor by a machine-learning process. In an embodiment, confidence training data may include a plurality of converted analog ECG data 116 correlated to examples of confidence intervals. Confidence training data may be received from database 300. confidence training data may contain information about digital ECG data 108, analog ECG data 116, normalized ECG data, examples of confidence intervals, and the like. In an embodiment, confidence training data may be iteratively updated as a function of the input and output results of past confidence machine-learning model or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, processor 104 is configured to generate an ECG validation set 124 as a function of the conversion of digital ECG data 108 to analog ECG data 116. As used in the current disclosure, an “ECG validation set” is a collection of data used to assess and validate the accuracy and performance of a particular algorithm, model, or method applied to ECG data. This ECG validation set 124 may be created by taking a portion of the analog ECG data 116, which has been converted from digital format, and using it for testing and validation purposes. The ECG dataset may be divided into three main subsets: the training set, the validation set, and the test set. The validation set is distinct from the training set (used to develop and train the algorithm or model) and the test set (used for final performance evaluation). The size of the ECG validation set 124 may vary depending on the specific goals of the validation process, but it may constitute a significant portion of the data, such as 20-30% of the dataset. This may provide enough data for meaningful validation while leaving ample data for training and testing. The ECG validation set 124 may be used to assess the performance of the algorithm or model in a controlled setting. It helps to identify issues such as overfitting (where the model performs well on the training data but poorly on new data), underfitting, and generalization errors. During the development of the algorithm or model, the ECG validation set 124 is used to fine-tune hyperparameters, optimize the model's performance, and make adjustments based on its performance on unseen data. In some cases, cross-validation techniques may be used with the ECG validation set 124, such as k-fold cross-validation. This may involve splitting the data into multiple subsets, repeatedly training on a subset of the data and validating on another subset, which helps in reducing the impact of random variations. The performance metrics of the machine learning model may be assessed using various metrics specific to the task, such as accuracy, sensitivity, specificity, or other ECG-specific metrics like QT interval accuracy for arrhythmia detection. Performance metrics are discussed in greater detail herein below. In some cases, the development and validation process may be often iterative. Adjustments may be made to the algorithm, model, or processing methods based on the feedback from the validation set. This process continues until the desired level of performance is achieved.

With continued reference to FIG. 1, the ECG validation set 124 may include intermittent ECG data. As used in the current disclosure, “intermittent ECG data” refers to a subset of the validation set that is composed of a plurality of ECG signals, where the signals are intermittently sampled from a range of values within the confidence interval. The ECG validation set 124 may include a subset of ECG signals that are used to assess the performance of an algorithm or model. It aims to evaluate how well the algorithm generalizes to unseen data. The confidence interval may represent a range of values within which the true ECG signal is expected to lie with a specified level of confidence. It quantifies the uncertainty associated with the ECG data and the conversion process from digital to analog. In the case of intermittent ECG data, ECG signals are not uniformly sampled throughout the entire confidence interval. Instead, they are intermittently sampled at specific points or intervals within the range. This approach introduces variability and uncertainty into the validation process. By intermittently sampling from the confidence interval, the validation set includes ECG signals that span the range of possible values within the interval, capturing both high and low points of uncertainty. Intermittent ECG data may provide diverse testing scenarios. It allows the evaluation of the algorithm's robustness to variations and fluctuations in the analog ECG signal caused by factors like noise, signal distortion, or inaccuracies in the conversion process. In some embodiments, assessing the algorithm's performance with intermittent ECG data, it may be possible to quantify its resilience and reliability under different levels of uncertainty and variability. Intermittent ECG data may be used to analyze the algorithm's sensitivity to different parts of the confidence interval, understanding how it responds to signals near the edges of the range compared to those in the middle. The performance of the algorithm or model is evaluated on this intermittent validation set using the same performance metrics as in traditional validation, but it may provide additional insights into the algorithm's behavior.

With continued reference to FIG. 1, processor 104 may generate an ECG validation set 124 using a validation machine-learning model 128. As used in the current disclosure, a “validation machine-learning model” is a machine-learning model that is configured to generate ECG validation set 124. Validation machine-learning model 128 may be consistent with the machine-learning model described below in FIG. 2. Inputs to the validation machine-learning model 128 may include analog ECG data 116, confidence interval, normalized ECG data, digital ECG data 108, examples of ECG validation sets 124, examples of intermittent ECG data, and the like. In some embodiments, inputs to the validation machine-learning model 128 may include analog ECG data 116 and one or more confidence intervals. Outputs to the validation machine-learning model 128 may include ECG validation set 124 tailored to the analog ECG data 116. Outputs to the validation machine-learning model 128 may additionally include intermittent ECG data tailored to the confidence interval of the analog ECG data 116. Validation training data may include a plurality of data entries containing a plurality of inputs that are correlated to a plurality of outputs for training a processor by a machine-learning process. In an embodiment, validation training data may include a plurality of analog ECG data 116 correlated to examples of ECG validation set 124. In another embodiment, validation training data may include the confidence interval of the analog ECG data 116 correlated to examples of intermittent ECG data. Validation training data may be received from database 300. validation training data may contain information about analog ECG data 116, confidence interval, normalized ECG data, digital ECG data 108, examples of ECG validation sets 124, examples of intermittent ECG data, and the like. In an embodiment, validation training data may be iteratively updated as a function of the input and output results of past validation machine-learning model 128 or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, machine learning plays a crucial role in enhancing the function of software for generating a validation machine-learning model 128. This may include identifying patterns of within the analog ECG data 116 or digital ECG data 108 that lead to changes in the capabilities and type of the validation machine-learning model 128. By analyzing vast amounts of data related to ECG signals, machine learning algorithms can identify patterns, correlations, and dependencies that contribute to a generating the validation machine-learning model 128. These algorithms can extract valuable insights from various sources, including text, document, EHRs, analog/digital ECG signals, and the like. By applying machine learning techniques, the software can generate the validation machine-learning model 128 extremely accurately. Machine learning models may enable the software to learn from past collaborative experiences of the entities and iteratively improve its training data over time.

With continued reference to FIG. 1, processor 104 may be configured to update the training data of the validation machine-learning model 128 using user inputs. A validation machine-learning model 128 may use user input to update its training data, thereby improving its performance and accuracy. In embodiments, the validation machine-learning model 128 may be iteratively updated using input and output results of past iterations of the validation machine-learning model 128. The validation machine-learning model 128 may then be iteratively retrained using the updated anonymization training data. For instance, and without limitation, validation machine-learning model 128 may be trained using first training data from, for example, and without limitation, training data from a user input or database. The validation machine-learning model 128 may then be updated by using previous inputs and outputs from the validation machine-learning model 128 as second training data to then train a second machine learning model. This process of updating the validation machine-learning model 128 and its associated training data may be continuously done to create subsequent validation machine-learning models 128 to improve the speed and accuracy of the validation machine-learning model 128. When users interact with the software, their actions, preferences, and feedback provide valuable information that can be used to refine and enhance the model. This user input is collected and incorporated into the training data, allowing the machine learning model to learn from real-world interactions and adapt its predictions accordingly. By continually incorporating user input, the model becomes more responsive to user needs and preferences, capturing evolving trends and patterns. This iterative process of updating the training data with user input enables the machine learning model to deliver more personalized and relevant results, ultimately enhancing the overall user experience. The discussion within this paragraph may apply to both the validation machine-learning model 128 or any other machine-learning model/classifier discussed herein.

Incorporating the user feedback may include updating the training data by removing or adding correlations of user data to a path or resources as indicated by the feedback. Any machine-learning model as described herein may have the training data updated based on such feedback or data gathered using a web crawler as described above. For example, correlations in training data may be based on outdated information wherein, a web crawler may update such correlations based on more recent resources and information.

With continued reference to FIG. 1, processor 104 may use user feedback to train the machine-learning models and/or classifiers described above. For example, machine-learning models and/or classifiers may be trained using past inputs and outputs of the machine-learning model. In some embodiments, if user feedback indicates that an output of machine-learning models and/or classifiers was “bad,” then that output and the corresponding input may be removed from training data used to train machine-learning models and/or classifiers, and/or may be replaced with a value entered by, e.g., another value that represents an ideal output given the input the machine learning model originally received, permitting use in retraining, and adding to training data; in either case, machine-learning models may be retrained with modified training data as described in further detail below. In some embodiments, training data of classifier may include user feedback.

With continued reference to FIG. 1, in some embodiments, an accuracy score may be calculated for the machine-learning model and/or classifier using user feedback. For the purposes of this disclosure, “accuracy score,” is a numerical value concerning the accuracy of a machine-learning model. For example, the accuracy/quality of the outputted validation machine-learning model 128 may be averaged to determine an accuracy score. In some embodiments, an accuracy score may be determined for pairing of entities. Accuracy score or another score as described above may indicate a degree of retraining needed for a machine-learning model and/or classifier. Processor 104 may perform a larger number of retraining cycles for a higher number (or lower number, depending on a numerical interpretation used), and/or may collect more training data for such retraining. The discussion within this paragraph and the paragraphs preceding this paragraph may apply to both the validation machine-learning model 128 or any other machine-learning model/classifier mentioned herein.

With continued reference to FIG. 1, processor 104 may generate a diagnostic machine-learning model 132. As used in the current disclosure, a “diagnostic machine-learning model 132” is a machine-learning model that is configured to generate diagnostic data. Diagnostic machine-learning model 132 may be consistent with the machine-learning model described below in FIG. 2. Inputs to the diagnostic machine-learning model 132 may include analog ECG data 116, normalized ECG data, digital ECG data 108, ECG validation sets 124, intermittent ECG data, and the like. Outputs to the diagnostic machine-learning model 132 may include diagnostic data. Diagnostic training data may include a plurality of data entries containing a plurality of inputs that are correlated to a plurality of outputs for training a processor by a machine-learning process. In an embodiment, diagnostic training data may include a plurality of analog ECG data 116 and/or normalized ECG data correlated to examples of diagnostic data. Diagnostic training data may be in an analog format and/or a digital format. In some embodiments, diagnostic training data may be retrieved from a database. Diagnostic training data may be received from database 300. Diagnostic training data may contain information about analog ECG data 116, confidence interval, normalized ECG data, digital ECG data 108, ECG validation sets 124, intermittent ECG data, examples of diagnostic data, and the like. In an embodiment, diagnostic training data may be iteratively updated as a function of the input and output results of past diagnostic machine-learning model 132 or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, the memory may instruct the processor to generate diagnostic data as a function of the acceptance of the diagnostic machine-learning model 132. As used in the current disclosure, “diagnostic data” is data associated with the diagnosis of a user's medical condition or health issue. Processor 104 may generate diagnostic data as a function of the collected and analyzed data from the ECG signals, this may include analog ECG data 116 and/or digital ECG data 108. Diagnostic data may identify the condition or the disease the user is suffering from based on known conditions/symptoms of the user associated with the ECG signals. In a non-limiting example, a abnormalities within an ECG signal may indicate the presence of abnormal rhythms (arrhythmias), such as atrial fibrillation or ventricular tachycardia. To generate the diagnostic data processor 104 may preprocess the ECG signals. The ECG signals may contain noise, baseline wander, and artifacts. Pre-processing steps involve filtering the signal to remove noise, such as powerline interference and muscle artifacts. The collected and preprocessed data may be typically stored in a secure database or storage system. This allows for easy retrieval and long-term archiving. Relevant features may be extracted from the ECG signals, such as peaks, trends, or statistical metrics. These features can help in characterizing the physiological parameter. In an embodiment, algorithms and machine learning techniques can be applied to recognize patterns and anomalies within the data. For instance, identifying irregular heart rhythms or unusual temperature fluctuations. The processor 104 may compare the collected ECG signals to reference data or established norms to assess whether the measurements fall within expected ranges. Processors 104 may use specialized diagnostic algorithms, which consider multiple parameters and historical data to make informed assessments. These algorithms can be based on clinical guidelines or specific domain knowledge. In some embodiments, if an abnormality or diagnostic insight is detected, the processor may generate a diagnostic report. This report typically may include information about the patient's condition, potential diagnoses, and recommended actions. If a critical condition or an emergency is detected, the processor can trigger alerts and notifications to healthcare providers, patients, or relevant stakeholders. In some embodiments, processor 104 may integrate the diagnostic data into a healthcare system, such as Electronic Health Records (EHR) systems, to share diagnostic information and ensure continuity of care.

With continued reference to FIG. 1, processor 104 may validate a diagnostic machine learning model 132 as a function of the ECG validation set 124. Validating a machine learning model may be an important step to ensure its reliability and effectiveness in making accurate predictions on new, unseen data. The process may involve assessing the model's performance and generalization capabilities, checking for overfitting or underfitting, and fine-tuning its parameters to optimize results. Validation may utilize a separate dataset (i.e. ECG validation set 124), distinct from the training data, to evaluate the model's ability to make predictions on examples it hasn't encountered during the learning phase. During validation, the model's performance may be assessed using various metrics, such as accuracy, precision, recall, F1 score, and area under the ROC curve, depending on the nature of the problem. These metrics may provide insights into the model's ability to correctly classify instances and avoid errors. Cross-validation techniques, like k-fold cross-validation, may also be employed to ensure robustness by training and validating the model on different subsets of the data. A well-validated machine learning model may demonstrate consistent and satisfactory performance across various evaluation metrics, instilling confidence in its ability to generalize to real-world scenarios and produce reliable predictions for the intended application. Validating a diagnostic machine learning model 132 involves assessing the model's performance and effectiveness in correctly diagnosing or classifying ECG data. This may include fine-tuning the model's hyperparameters using the ECG validation set 124. Fine-tuning the model's hyperparameters may include adjusting learning rates, regularization techniques, and other parameters to optimize the model's performance.

With continued reference to FIG. 1, processor 104 is configured to generate performance data 136 associated with the diagnostic learning model 132 based on the ECG validation set 124. As used in the current disclosure, “performance data” is data that provides insights into how well the model is functioning in terms of accuracy, precision, recall, and other relevant metrics. These metrics are crucial for assessing the model's ability to correctly classify instances, avoid errors, and generalize to new, unseen data. Performance data 136 may include data associated with the accuracy of the diagnostic learning model 132. Accuracy may represent the proportion of correctly classified instances among all instances. It is a general measure of the model's overall correctness and is calculated as the ratio of true positives and true negatives to the total number of instances. Performance data 136 may include data associated with the precision of the diagnostic learning model 132. Precision, may be referred to as a positive predictive value, may measure the accuracy of positive predictions made by the model. It may be calculated as the ratio of true positives to the sum of true positives and false positives, providing insights into the model's ability to avoid false positives. Performance data 136 may include recall, or sensitivity, measurements for the machine learning model. Recall measures the proportion of actual positive instances that the model correctly identifies. It may be calculated as the ratio of true positives to the sum of true positives and false negatives, indicating the model's ability to avoid false negatives. Performance data 136 may include the generation of a F1 Score. The F1 score may be a harmonic mean of precision and recall, providing a balance between the two metrics. It is particularly useful when there is an imbalance between the number of positive and negative instances in the dataset. Performance data 136 may include specificity, wherein specificity may measure the proportion of actual negative instances that the model correctly identifies. Specificity may be measured as the ratio of true negatives to the sum of true negatives and false positives. Performance data 136 may include a measurement of the Area Under the ROC Curve (AUC-ROC). The ROC curve may include a visualization of the trade-off between true positive rate (sensitivity) and false positive rate. The AUC-ROC summarizes the overall performance of the model across different classification thresholds. Performance data 136 may include the generation of a confusion matrix. The confusion matrix may provide a detailed breakdown of true positives, true negatives, false positives, and false negatives. It is a valuable tool for understanding the distribution of prediction outcomes. Performance data 136 may include a prediction of the accuracy at Top-K. In scenarios where only the top predictions are considered (e.g., top 3 or top 5 predictions), accuracy at the top-K may provide insights into the model's performance when multiple predictions are allowed. Performance data 136 may include a measurement of the Area Under the Precision-Recall Curve (AUC-PR). The precision-recall curve may include a visualization of the trade-off between precision and recall at different classification thresholds. AUC-PR summarizes the model's performance in scenarios where positive instances are rare. Performance data 136 may include a measurement of the calibration curve. The calibration curve may assess the alignment between predicted probabilities and actual outcomes. A well-calibrated model may produce predicted probabilities that accurately reflect the likelihood of positive instances.

With continued reference to FIG. 1, performance data 136 may include error analysis. As used in the current disclosure, “error analysis” is an analysis of the mistakes or inaccuracies made by a model during its predictions. It may be a step in identifying the limitations of the model, uncovering patterns in misclassifications, and informing improvements to enhance the model's performance. Analyzing patterns in errors may help uncover specific challenges or weaknesses in the model. For instance, the model may struggle with certain classes, specific types of input data, or cases with particular characteristics. Error analysis may begin by identifying instances where the model has made mistakes. This involves reviewing the predictions and comparing them with the true outcomes in the dataset. Error analysis may include an analysis of the importance of different features or variables within the ECG validation set 124. This may include misclassified instances and insights into which aspects of the data are more challenging for the model to accurately predict. Error analysis may include data associated the distribution of errors across different subsets of the data, such as specific demographics or subgroups. This data may help in identifying areas where the model may need improvement or where additional training data may be beneficial. The findings from error analysis may guide the iterative process of model improvement. Adjustments to the model architecture, feature engineering, or the collection of additional training data can be informed by the specific challenges identified through error analysis. In some instances, error analysis is not a one-time process; it should be part of an ongoing monitoring strategy. As new data becomes available, the model's performance should be regularly evaluated, and error analysis should be conducted to identify any emerging issues.

With continued reference to FIG. 1, performing an error analysis may include generation of a confusion matrix. As used in the current disclosure, a “confusion matrix” is a table that is used to evaluate the performance of a classification algorithm. A confusion matrix may include the identification various error types, wherein error types may include false positives (FP), false negatives (FN), true positives (TP), and true negatives (TN). False Positives may be instances where the model predicted a positive outcome, but the true outcome was negative. False Negatives may be instances where the model predicted a negative outcome, but the true outcome was positive. True positives and true negatives may be instances where the model correctly predicted a positive/negative outcome associated with the diagnostic learning model 132. The confusion matrix may provide a breakdown of true positives, true negatives, false positives, and false negatives. This matrix offers a clear overview of the model's performance and the types of errors it tends to make.

With continued reference to FIG. 1, processor 104 may be configured to identify error patterns as a function of the confusion matrix. An error pattern may refer to a consistent and identifiable trend or type of mistake made by a model when predicting outcomes in a machine learning task. It may involve understanding the nature of errors that the model tends to make and can be revealed by examining the confusion matrix or other evaluation metrics. In a non-limiting example, processor 104 may generate an error pattern by examining instances where the model made incorrect predictions and/or correct predictions. The processor 104 may then identify patterns and/or commonalities in the errors. This may involve reviewing misclassified samples, exploring false positives, and understanding false negatives. Error patterns may be described based on the accuracy, precision, recall, specificity, F1 score, false positive rate, false negative rate, and the like of the diagnostic machine learning model. In an embodiment, the overall correctness of the model may be calculated as (TP+TN)/(TP+FP+FN+TN). It may give the proportion of correctly classified instances. In another embodiment, the precision of the model may be calculated as TP/(TP+FP). It represents the accuracy of positive predictions. The rate of false positives may be calculated as FP/(FP+TN). It measures the proportion of actual negatives that are incorrectly predicted as positive. Alternatively, the rate of false negatives may be calculated as FN/(FN+TP). It measures the proportion of actual positives that are incorrectly predicted as negatives.

With continued reference to FIG. 1, the results of the error analysis may be presented to a user in an error report. As used in the current disclosure, an “error report” is a report that includes details about the types of errors, patterns observed, and actions taken for improvement. It serves as a reference for ongoing model maintenance and updates. An error report may be generated as a function of error analysis, the report may serve as a detailed document summarizing the findings, insights, and recommendations derived from a thorough examination of a machine learning model's mistakes or inaccuracies. The error report may briefly summarize the key findings of the error analysis, including high-level insights into the model's performance, major challenges identified, and overarching recommendations. The error report may discuss the types of errors identified, such as false positives and false negatives. The report may additionally highlight any patterns or trends observed in the misclassifications, such as specific classes that are challenging for the model or certain characteristics of misclassified instances. This may include an analysis of how errors are distributed across different subsets of the data, such as demographic groups or specific conditions. Additionally, the report may include an identification of areas where the model may perform differently or encounter challenges. The report may provide context for the error report, outlining the goals and objectives of the model, the dataset used, and the specific tasks the model is designed to perform. The error report may present the confusion matrix detailing true positives, true negatives, false positives, and false negatives. The error report may include various performance data 136 such as accuracy, precision, recall, F1 score, specificity, area under the ROC curve, and any other relevant metrics. In some cases, the error report may provide specific recommendations for enhancing the model's performance based on the identified challenges. This may include adjustments to the model architecture, additional feature engineering, or the collection of targeted training data. The report may include details about any changes or updates made to the model as a result of the error analysis. This serves as a historical record and helps track the model's evolution over time. In some cases, the error report may be presented to a user who is a domain expert. Collaborating with domain experts, such as medical professionals in the case of diagnostic models, can provide valuable insights into the clinical relevance of specific errors and contribute to refining the model.

With continued reference to FIG. 1, the processor 104 is configured to compare the performance data 136 to a validation threshold 140. As used in the current disclosure, a “validation threshold” refers to a predefined criterion or set of criteria that determines whether a machine learning model is accepted or rejected based on its performance data. This threshold may be established by comparing one or more performance data 136, such as accuracy, precision, recall, or F1 score, against predefined acceptable values. The validation threshold serves as a decision rule, guiding whether the model meets the desired level of performance for deployment or requires further refinement. Processor 104 may define acceptable ranges or values for the validation threshold 140 as a function of the selected performance data 136. These thresholds are often determined based on the requirements of the application, industry standards, or regulatory guidelines. In some cases, validation thresholds 140 may be established as a function of input from domain experts who have knowledge in the specific field related to the machine learning task. Their insights can help define realistic expectations and acceptable ranges for performance metrics. While generating the validation threshold 140, processor 104 may consider trade-offs between different metrics. For example, in binary classification, increasing precision might lead to a decrease in recall and vice versa. The processor 104 may establish a balance that aligns with the task requirements. In some embodiments, processor 104 may simulate the impact of different validation threshold 140 values on the machine learning model's predictions. This allows for an understanding of how changes in the validation threshold 140 may affect the trade-offs between true positives, true negatives, false positives, and the like.

With continued reference to FIG. 1, processor 104 may establish a decision rule based on the comparison results. As used in the current discourse, a “decision rule” refers to a predefined criterion or set of conditions used to make a decision based on the output of a model. It may be a guiding principle that helps determine the action or classification assigned to a particular instance based on the model's predictions. Decision rules are essential for translating the continuous or probabilistic outputs of a model into actionable outcomes or classifications. A decision rule often may involve applying a threshold to the model's output. For example, in binary classification, a threshold might be set at 0.5 for probability scores. Instances with a probability above 0.5 are classified as positive, and those below 0.5 are classified as negative. Once the threshold is applied, the decision rule may determine the assigned classification or action. For binary classification, this might be assigning a class label (e.g., positive or negative). In regression tasks, it could involve categorizing the output into predefined ranges. In multiclass classification, decision rules may extend to assigning instances to one of multiple classes. This may involve selecting the class with the highest probability or considering additional criteria to break ties.

With continued reference to FIG. 1, after training the machine learning model, processor 104 may compare historical performance metrics to the predefined validation threshold to identify a validation threshold 140. This involves assessing whether the model's performance falls within the acceptable range for each metric as detailed by the validation threshold 140. For example, if the model's performance meets or exceeds the validation threshold for all relevant metrics, the model is accepted. Conversely if the performance falls below the threshold for any critical metric, further analysis, refinement, or iteration may be necessary. If the model does not meet the validation threshold, the processor 104 may implement additional training cycles on the model, adjusting hyperparameters, refining features, or incorporating additional training data. The validation threshold 140 may serve as a benchmark for iterative improvements. In certain applications, the validation threshold 140 may also take into account the business or clinical impact of model's performance. For example, a diagnostic model for a life-threatening condition may have stricter performance requirements than a model for a less critical task.

With continued reference to FIG. 1, accepting a diagnostic machine learning model may involve a comprehensive evaluation and decision-making process to determine whether the model meets the predefined criteria for deployment and use in a real-world setting. The acceptance decision is typically made based on the model's performance, reliability, and alignment with specific requirements and objectives. Processor 104 may establish clear and measurable acceptance criteria based on the goals and requirements of the diagnostic machine learning model. These criteria may include specific performance metrics (e.g., accuracy, precision, recall), regulatory compliance, and alignment with clinical or business objectives. The model may be accepted based on the model's performance using a dedicated validation set or test set that was not used during training. Processor 104 may evaluate relevant metrics to ensure that the model achieves the desired level of accuracy and reliability. This may include a consideration the impact of false positives and false negatives in the context of the diagnostic application. In medical applications, acceptance of the model may involve healthcare professionals and domain experts. Validating the model's outputs against established medical knowledge, guidelines, and, if available, independent clinical assessments. Ensure that the model's predictions align with expected clinical outcomes. In some cases, acceptance of the model may include verifying that the model complies with relevant regulations and standards in the healthcare or diagnostic domain. This may involve adhering to data privacy regulations, obtaining necessary approvals from regulatory bodies, and ensuring the model meets industry-specific guidelines.

Still referring to FIG. 1, processor 104 may be configured to display the results of the validation of the diagnostic machine-learning model 132 using a display device 144. As used in the current disclosure, a “display device” is a device that is used to display a plurality of data and other digital content. A display device 144 may include a user interface. A “user interface,” as used herein, is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof, and the like. A user interface may include a smartphone, smart tablet, desktop, or laptop operated by the user. In an embodiment, the user interface may include a graphical user interface. A “graphical user interface (GUI),” as used herein, is a graphical form of user interface that allows users to interact with electronic devices. In some embodiments, GUI may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pulldown menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access. Information contained in user interface may be directly influenced using graphical control elements such as widgets. A “widget,” as used herein, is a user control element that allows a user to control and change the appearance of elements in the user interface. In this context a widget may refer to a generic GUI element such as a check box, button, or scroll bar to an instance of that element, or to a customized collection of such elements used for a specific function or application (such as a dialog box for users to customize their computer screen appearances). User interface controls may include software components that a user interacts with through direct manipulation to read or edit information displayed through user interface. Widgets may be used to display lists of related items, navigate the system using links, tabs, and manipulate data using check boxes, radio boxes, and the like.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example analog ECG data 116 as inputs correlated to ECG validation sets 124 as outputs.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to analog ECG data 116 associated with known demographic and/or condition.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may identified as having poor quality data, where “poor quality” is defined as having a signal to noise ratio below a threshold value.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include analog ECG data 116 as described above as inputs, ECG validation sets 124 as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

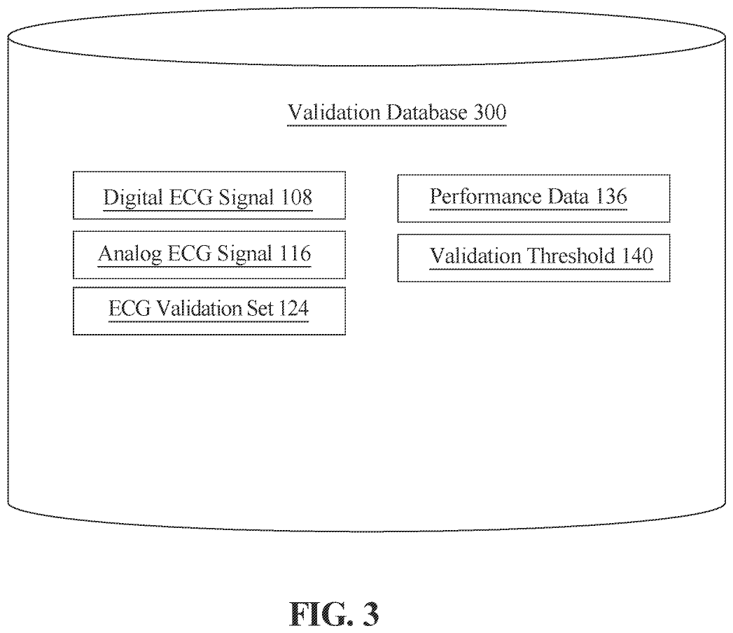

Now referring to FIG. 3, an exemplary validation database 300 is illustrated by way of block diagram. In an embodiment, any past or present versions of any data disclosed herein may be stored within the validation database 300 including but not limited to: analog ECG data 116, confidence intervals, normalized ECG data, digital ECG data 108, ECG validation sets 124, intermittent ECG data, diagnostic data, performance data 136, validation thresholds 140, and the like. Processor 104 may be communicatively connected with validation database 300. For example, in some cases, database 300 may be local to processor 104. Alternatively or additionally, in some cases, database 300 may be remote to processor 104 and communicative with processor 104 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 104 connects directly, dynamically, and non-hierarchically to as many other computing devices as possible. A “network topology” as used in this disclosure is an arrangement of elements of a communication network. validation database 300 may be implemented, without limitation, as a relational database, a key-value retrieval database such as a NOSQL database, or any other format or structure for use as a database that a person skilled in the art would recognize as suitable upon review of the entirety of this disclosure. validation database 300 may alternatively or additionally be implemented using a distributed data storage protocol and/or data structure, such as a distributed hash table or the like. validation database 300 may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

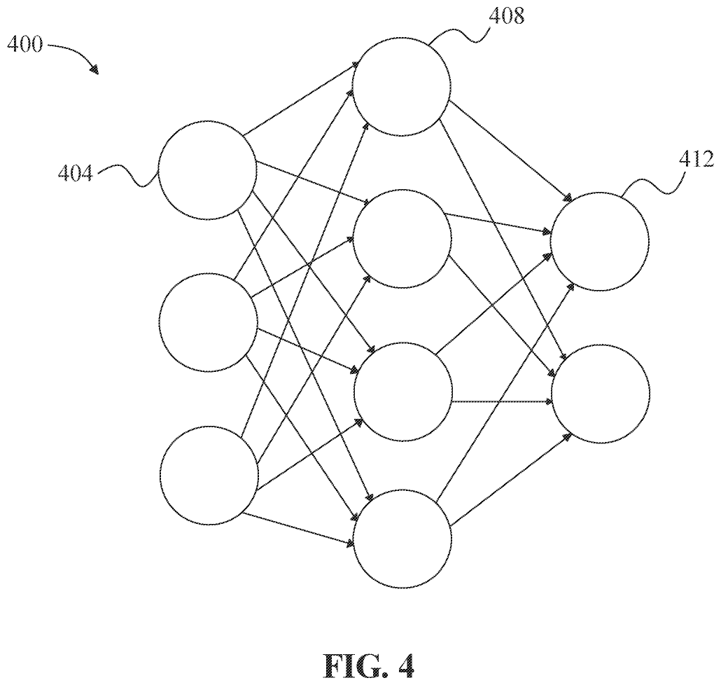

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

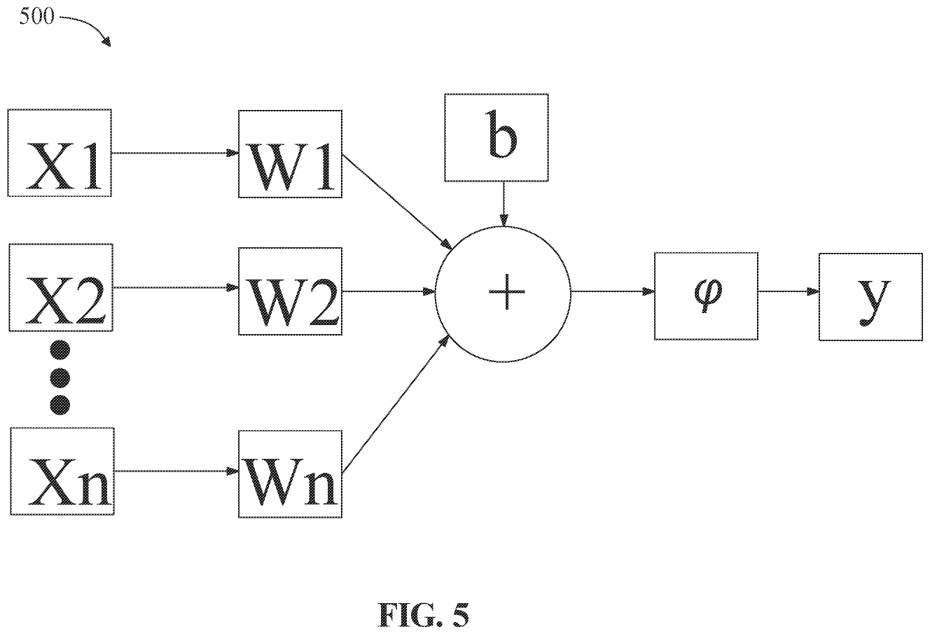

Referring now to FIG. 5, an exemplary embodiment of a node of a neural network is illustrated. A node may include, without limitation, a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

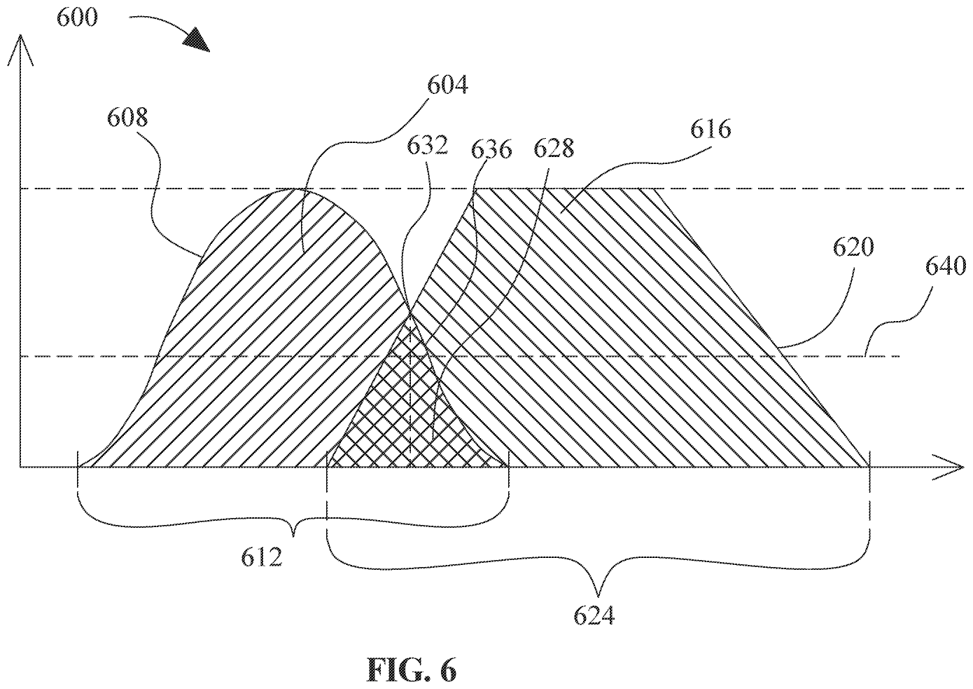

Now referring to FIG. 6, an exemplary embodiment of fuzzy set comparison 600 is illustrated. In a non-limiting embodiment, the fuzzy set comparison. In a non-limiting embodiment, fuzzy set comparison 600 may be consistent with fuzzy set comparison in FIG. 1. In another non-limiting the fuzzy set comparison 600 may be consistent with the name/version matching as described herein. For example and without limitation, the parameters, weights, and/or coefficients of the membership functions may be tuned using any machine-learning methods for the name/version matching as described herein. In another non-limiting embodiment, the fuzzy set may represent a performance data 136 and a validation threshold 140 from FIG. 1.

Alternatively or additionally, and still referring to FIG. 6, fuzzy set comparison 600 may be generated as a function of determining the data compatibility threshold. The compatibility threshold may be determined by a computing device. In some embodiments, a computing device may use a logic comparison program, such as, but not limited to, a fuzzy logic model to determine the compatibility threshold and/or version authenticator. Each such compatibility threshold may be represented as a value for a posting variable representing the compatibility threshold, or in other words a fuzzy set as described above that corresponds to a degree of compatibility and/or allowability as calculated using any statistical, machine-learning, or other method that may occur to a person skilled in the art upon reviewing the entirety of this disclosure. In some embodiments, determining the compatibility threshold and/or version authenticator may include using a linear regression model. A linear regression model may include a machine learning model. A linear regression model may map statistics such as, but not limited to, frequency of the same range of version numbers, and the like, to the compatibility threshold and/or version authenticator. In some embodiments, determining the compatibility threshold of any posting may include using a classification model. A classification model may be configured to input collected data and cluster data to a centroid based on, but not limited to, frequency of appearance of the range of versioning numbers, linguistic indicators of compatibility and/or allowability, and the like. Centroids may include scores assigned to them such that the compatibility threshold may each be assigned a score. In some embodiments, a classification model may include a K-means clustering model. In some embodiments, a classification model may include a particle swarm optimization model. In some embodiments, determining a compatibility threshold may include using a fuzzy inference engine. A fuzzy inference engine may be configured to map one or more compatibility threshold using fuzzy logic. In some embodiments, a plurality of computing devices may be arranged by a logic comparison program into compatibility arrangements. A “compatibility arrangement” as used in this disclosure is any grouping of objects and/or data based on skill level and/or output score. Membership function coefficients and/or constants as described above may be tuned according to classification and/or clustering algorithms. For instance, and without limitation, a clustering algorithm may determine a Gaussian or other distribution of questions about a centroid corresponding to a given compatibility threshold and/or version authenticator, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Still referring to FIG. 6, inference engine may be implemented according to input performance data 136 and validation threshold 140. For instance, an acceptance variable may represent a first measurable value pertaining to the classification of performance data 136 to validation threshold 140. Continuing the example, an output variable may represent an accepted diagnostic machine-learning model 132. In an embodiment, performance data 136 and/or validation threshold 140 may be represented by their own fuzzy set. In other embodiments, the acceptance of the diagnostic machine-learning model 132 may be represented as a function of the intersection two fuzzy sets as shown in FIG. 6, An inference engine may combine rules, such as any semantic versioning, semantic language, version ranges, and the like thereof. The degree to which a given input function membership matches a given rule may be determined by a triangular norm or “T-norm” of the rule or output function with the input function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥,” such as max (a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$( x , a , b , c ) = \{ \begin{matrix}0 , & for x > c and x < a \\ \frac{x - a}{b - a} , & for a \leq x < b \\ \frac{c - x}{c - b} , & if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

First fuzzy set 604 may represent any value or combination of values as described above, including any performance data 136 and validation threshold 140. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 636 that overlaps, first membership function 608 and second membership function 620 may intersect at a point 632 representing a probability, as defined on probability interval, of a match between first fuzzy set 604 and second fuzzy set 616. Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, the classification into one or more query categories may indicate a sufficient degree of overlap with fuzzy set representing performance data 136 and validation threshold 140 for combination to occur as described above. Each threshold may be established by one or more user inputs. Alternatively or additionally, each threshold may be tuned by a machine-learning and/or statistical process, for instance and without limitation as described in further detail below.

In an embodiment, a degree of match between fuzzy sets may be used to rank one resource against another. For instance, if both performance data 136 and validation threshold 140 have fuzzy sets, Acceptance of the diagnostic machine-learning model 132 may be done by having a degree of overlap exceeding a predictive threshold, processor 104 may further rank the two resources by ranking a resource having a higher degree of match more highly than a resource having a lower degree of match. Where multiple fuzzy matches are performed, degrees of match for each respective fuzzy set may be computed and aggregated through, for instance, addition, averaging, or the like, to determine an overall degree of match, which may be used to rank resources; selection between two or more matching resources may be performed by selection of a highest-ranking resource, and/or multiple notifications may be presented to a user in order of ranking.

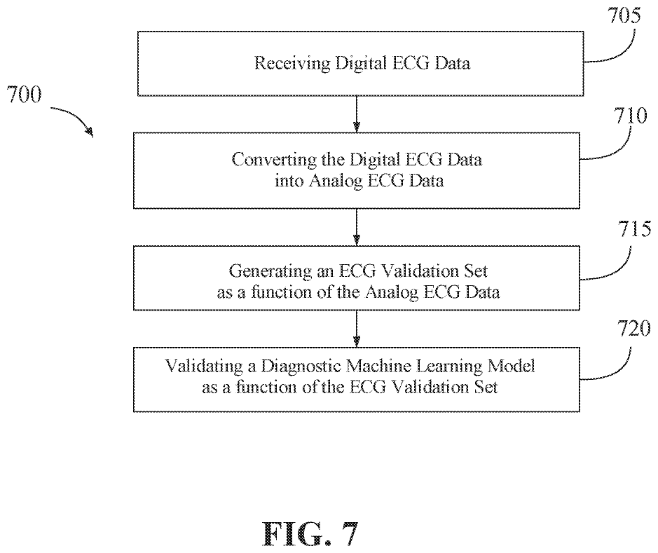

Referring now to FIG. 7, a flow diagram of an exemplary method 700 for generating an electrocardiogram verification set is illustrated. At step 705, method 700 includes receiving, using at least a processor, digital ECG data. This may be implemented as described and with reference to FIGS. 1-7. In an embodiment, the method may further include anonymizing, using the at least a processor, the digital ECG data using an anonymization process. In additional embodiment, the ECG validation set may be generated using a validation machine learning model.

Still referring to FIG. 7, at step 710, method 700 includes converting, using the at least a processor, the digital ECG data into analog ECG data. This may be implemented as described and with reference to FIGS. 1-7. In an embodiment, converting the plurality of digital ECG data may include converting the plurality of digital ECG data into analog ECG data using a Digital-to-Analog Converter. In an embodiment, the method further may further include generating, using the at least a processor, normalized ECG data as a function of the conversion. In another embodiment, wherein the method may further include calculating, using the at least a processor, a confidence interval associated with the conversion of the analog ECG data. The ECG validation set may include intermittent ECG data, wherein the intermittent of ECG data includes a plurality of ECG signals intermittently sampled from a range of values within the confidence interval.

Still referring to FIG. 7, at step 715, method 700 includes generating, using the at least a processor, an ECG validation set as a function of the analog ECG data. This may be implemented as described and with reference to FIGS. 1-7.

Still referring to FIG. 7, at step 720, method 700 includes validating, using the at least a processor, a diagnostic machine learning model as a function of the ECG validation set. Validating the diagnostic machine learning model includes iteratively training the diagnostic machine learning model using diagnostic training data. Validating the diagnostic machine learning model includes generating performance data based on the ECG validation set. Validating the diagnostic machine learning model includes accepting the diagnostic machine learning model as a function of a comparison between the performance data and a validation threshold. This may be implemented as described and with reference to FIGS. 1-7. In an embodiment, generating the performance data may include performing an error analysis on the diagnostic machine learning model. The method may further include generating, using the at least a processor, an error report as a function of the error analysis. In another embodiment, the method may further include iteratively retraining, using the at least a processor, the diagnostic machine learning model as function of the performance data failing to meet the validation threshold.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

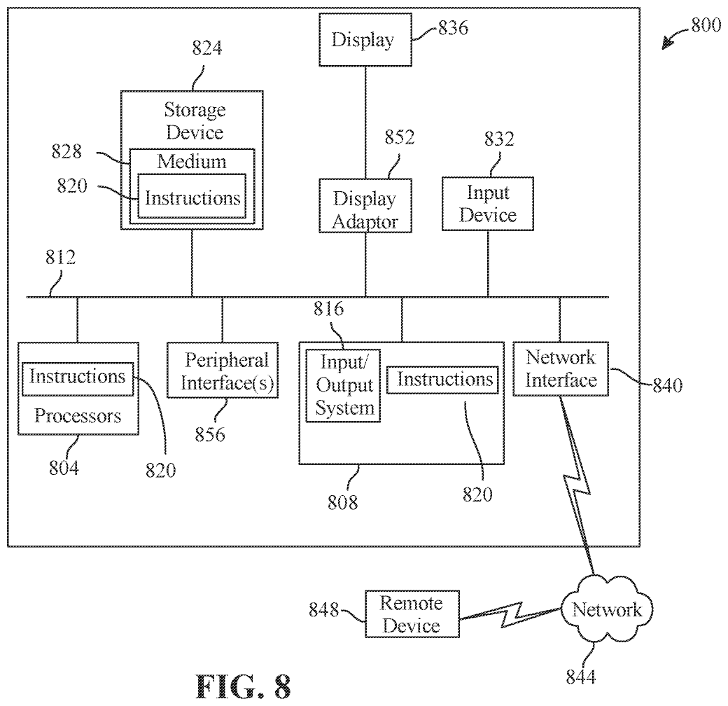

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for generating an electrocardiogram (ECG) verification set, wherein the apparatus comprises:
at least a processor; and
a memory communicatively connected to the at least a processor, wherein the memory contains instructions configuring the at least a processor to:
receive digital ECG data;
convert the digital ECG data into analog ECG data;
generate an ECG validation set, diagnostic training data, and a test set by dividing the analog ECG data utilizing a validation machine learning model, wherein generating the ECG validation set further comprises:
receiving validation training data, wherein the validation training data comprises inputs of analog ECG data correlated to outputs of ECG validation sets;
training, iteratively, the validation machine learning model using the validation training data, wherein training the validation machine learning model includes retraining the validation machine learning model with feedback from previous iterations of the validation machine learning model; and
generating the ECG validation set using the trained validation machine learning model; and
validate a diagnostic machine learning model as a function of the ECG validation set, wherein validating the diagnostic machine learning model comprises:
iteratively training the diagnostic machine learning model using diagnostic training data;
inputting the ECG validation set into the diagnostic machine learning model;
generating performance data associated with the diagnostic machine learning model based on the ECG validation set;
comparing the performance data to a validation threshold;
retraining the diagnostic machine learning model as a function of the comparison of the performance data to the validation threshold, wherein retraining the diagnostic machine learning model comprises incorporating additional ECG data in the diagnostic training data; and
accepting the diagnostic machine learning model as a function of the test set; and
generate diagnostic data as a function of accepting the diagnostic machine learning model, wherein the diagnostic data comprises at least an abnormality.

2. The apparatus of claim 1, wherein converting the digital ECG data comprises converting the digital ECG data into the analog ECG data using a digital-to-analog converter.

3. The apparatus of claim 1, wherein the memory further instructs the at least a processor to generate normalized ECG data as a function of the conversion of the digital ECG data into the analog ECG data.

4. The apparatus of claim 1, wherein the memory further instructs the at least a processor to calculate one or more confidence intervals associated with the conversion of the digital ECG data into the analog ECG data.

5. The apparatus of claim 4, wherein the ECG validation set comprises intermittent ECG data, wherein the intermittent ECG data comprises a plurality of ECG signals intermittently sampled from a range of values within the one or more confidence intervals.

6. The apparatus of claim 1, wherein generating the performance data comprises performing an error analysis on the diagnostic machine learning model, wherein performing the error analysis comprises:
generating a confusion matrix as a function of an output of the diagnostic machine learning model; and
identifying one or more error patterns associated with the confusion matrix.

7. The apparatus of claim 6, wherein the memory further instructs the at least a processor to generate an error report as a function of the error analysis.

8. The apparatus of claim 1, wherein the memory further instructs the at least a processor to iteratively retrain the diagnostic machine learning model as a function of the performance data failing to meet the validation threshold.

9. The apparatus of claim 1, wherein the memory further instructs the at least a processor to anonymize the digital ECG data using an anonymization process.

10. (canceled)

11. A method for generating an electrocardiogram (ECG) verification set, wherein the method comprises:
receiving, using at least a processor, digital ECG data;
converting, using the at least a processor, the digital ECG data into analog ECG data;
generating, using the at least a processor, an ECG validation set, diagnostic training data, and a test set by dividing the analog ECG data utilizing a validation machine learning model, wherein generating the ECG validation set further comprises:
receiving validation training data, wherein the validation training data comprises inputs of analog ECG data correlated to outputs of ECG validation sets;
training, iteratively, the validation machine learning model using the validation training data, wherein training the validation machine learning model includes retraining the validation machine learning model with feedback from previous iterations of the validation machine learning model;
generating the ECG validation set using the trained validation machine learning model; and
validating, using the at least a processor, a diagnostic machine learning model as a function of the ECG validation set, wherein validating the diagnostic machine learning model comprises:
iteratively training the diagnostic machine learning model using diagnostic training data;
inputting the ECG validation set into the diagnostic machine learning model;
generating performance data associated with the diagnostic machine learning model based on the ECG validation set;
comparing the performance data to a validation threshold;
retraining the diagnostic machine learning model as a function of the comparison of the performance data to the validation threshold, wherein retraining the diagnostic machine learning model comprises incorporating additional ECG data in the diagnostic training data; and
accepting the diagnostic machine learning model as a function of the test set; and
generating, using the at least a processor, diagnostic data as a function of accepting the diagnostic machine learning model, wherein the diagnostic data comprises at least an abnormality.

12. The method of claim 11, wherein converting the digital ECG data comprises converting the digital ECG data into the analog ECG data using a digital-to-analog converter.

13. The method of claim 11, wherein the method further comprises generating, using the at least a processor, normalized ECG data as a function of the conversion of the digital ECG data into the analog ECG data.

14. The method of claim 11, wherein the method further comprises calculating, using the at least a processor, one or more confidence intervals associated with the conversion of the digital ECG data into the analog ECG data.

15. The method of claim 14, wherein the ECG validation set comprises intermittent ECG data, wherein the intermittent ECG data comprises a plurality of ECG signals intermittently sampled from a range of values within the one or more confidence intervals.

16. The method of claim 11, wherein generating the performance data comprises performing an error analysis on the diagnostic machine learning model, wherein performing the error analysis comprises:
generating a confusion matrix as a function of an output of the diagnostic machine learning model; and
identifying one or more error patterns associated with the confusion matrix.

17. The method of claim 16, wherein the method further comprises generating, using the at least a processor, an error report as a function of the error analysis.

18. The method of claim 11, wherein the method further comprises iteratively retraining, using the at least a processor, the diagnostic machine learning model as a function of the performance data failing to meet the validation threshold.

19. The method of claim 11, wherein the method further comprises anonymizing, using the at least a processor, the digital ECG data using an anonymization process.

20. (canceled)
